(19) United States
(12) Patent Application Publication
(10) Pub. No.: US 2020/0297847 A1
(43) Pub. Date: Sep. 24, 2020

(54) Pharmaceutical Composition For Treating Obesity Induced By High Fat Diet And Non-Alcoholic Fatty Liver Disease By Using Trisodium Chlorin E6 Photosensitizer

(71) Applicant: DONG SUNG PHARM. CO., LTD., Seoul (KR)

(72) Inventors: Yong Wan KIM, Seoul (KR); Anil Kumar CHAUHAN, Daegu (KR)

(21) Appl. No.: 16/896,443

(22) Filed: Jun. 9, 2020

Related U.S. Application Data
(63) Continuation of application No. PCT/KR2018/015722, filed on Dec. 11, 2018.

(30) Foreign Application Priority Data
Dec. 11, 2017 (KR) 10-2017-0169338

(51) Int. Cl. A61K 41/00; A61P 3/04

(52) U.S. Cl. CPC A61K 41/0071; A61K 9/0019; A61P 3/04

(57) ABSTRACT

Disclosed is a pharmaceutical composition for obesity inhibition for photodynamically inhibiting obesity, containing trisodium Chlorin e6 as an active ingredient.

### CROSS-REFERENCE TO RELATED APPLICATIONS

This application is a continuation of PCT Application No. PCT/KR2018/015722, filed on 11 Dec. 2018, which claims benefit of Korean Patent Application 10-2017-0169338, filed on 11 Dec. 2017. The entire disclosure of the applications identified in this paragraph is incorporated herein by references.

### FIELD

The present invention relates to a pharmaceutical composition for the treatment of obesity induced by a high-fat diet and non-alcoholic steatohepatitis using a trisodium Chlorin e6 photosensitizer and to an optical treatment method.

### BACKGROUND

Obesity is increasing in developed countries. Approximately 23% of adult Canadians (5.5 million people) are obese (defined as a body mass index [BMI] of 30 kg/m² or more) and an additional 36% are overweight (BMI of 25 kg/m² or more) [Reference 1], compared with an obesity rate of 14% in the late 1970s. The causes of increased obesity may be multifactorial. Although there is certainly a genetic predisposition to obesity, various environmental factors are also implicated, including over-intake, dietary macronutrient composition and sedentary lifestyles provided by modern amenities [Reference 2].

Obesity-related complications directly incur significant medical costs of $ 1.8 billion per year, which is approximately 2.4% of all medical expenditure [Reference 3]. It is also associated with increased risk for multiple diseases: coronary artery disease, cerebrovascular disease, hypertension, type 2 hyperlipidemia, diabetes, cholelithiasis, pulmonary embolism, sleep apnea, gynecological abnormalities, degenerative arthritis, psychiatric illness and malignant tumors (breast, endometrial, prostate and colon). Moreover, it is an independent risk factor for increased mortality [Reference 3].

Obesity has major health implications in gastroenterology because it plays a main role in the pathogenesis of non-alcoholic fatty liver disease (hereinafter, ‘NAFLD’). NAFLD is the most common cause of abnormal liver tests in North America, with a prevalent of 32% in obese males and 42% in obese females [Reference 4]. Disease pathogenesis is related to insulin resistance and oxidative stress.

NAFLD is no longer regarded as a benign condition, and has the potential to manifest as steatosis, steatohepatitis or fibrosis and then progresses to cirrhosis in 5% of patients within 7 years [References 5 and 6]. The natural history of NAFLD indicates that the presence of fat in liver biopsies is associated with reduced life expectancy [Reference 6]. The risk factors for NAFLD include obesity (67% to 71% of patients), glucose intolerance (12% to 37% of patients), dyslipidemia (57% to 68% of patients), and hypertension (36% to 70% of patients). Moderate weight loss (5% to 10%) in these people may significantly reduce obesity-related disorders and complications [Reference 8].

Non-alcoholic fatty liver disease (NAFLD) is a generic term that encompasses not only the simple accumulation of adipose tissue in the liver but also hepatitis, fibrosis, cirrhosis and, in some cases, more advanced steatosis associated with hepatocellular carcinoma (hereinafter ‘HCC’) [Reference 9]. The term ‘NAFLD’ encompasses both non-alcoholic fatty liver (hereinafter, ‘NAFL’) and non-alcoholic steatohepatitis (hereinafter, ‘NASH’). [Reference 9]. NAFL is defined by the presence of steatosis, with 5% or more of liver parenchyma involved and no evidence of hepatocellular injury [Reference 10]. Compared thereto, NASH is a histologically defined degenerative inflammatory disease, and hepatocytes are damaged due to hepatitis [Reference 10]. The natural history of NAFLD has not been fully elucidated, but what is evident from the published data is the risk of progressing to cirrhosis and HCC [References 11-15]. In view of current irresponsible eating habits and the prevalence of a sedentary lifestyle, it is not surprising that NAFLD has spread worldwide in recent years [9]. Moreover, NAFLD has also increased in proportion with the spread of diabetes and metabolic syndrome [11]. A US study has found that NAFLD is 10% more prevalent in overweight people than in skinny people. Indeed, NAFLD will not only be the leading cause of liver-associated morbidity and mortality over the next 20 years, but will also be a leading indication of liver transplantation [Reference 11].

In this regard, efforts have been made to develop appropriate treatments for obesity and obesity-related diseases, but effective treatment methods are still lacking to date.

### SUMMARY

### Technical Problem

An aspect of the present invention is to provide a pharmaceutical composition for the treatment of obesity and non-alcoholic steatohepatitis and an optical treatment method.

Another aspect of the present invention is to provide a pharmaceutical composition for photodynamic treatment of obesity, containing trisodium Chlorin e6 as an active ingredient, and a photodynamic treatment method using the same.

Still another aspect of the present invention is to provide a pharmaceutical composition for photodynamic treatment of non-alcoholic steatohepatitis, containing trisodium Chlorin e6 as an active ingredient, and a photodynamic treatment method using the same.

### Technical Solution

An embodiment of the present invention provides a pharmaceutical composition for obesity inhibition for photodynamically inhibiting obesity, containing trisodium Chlorin e6 as an active ingredient.

Another embodiment of the present invention provides a pharmaceutical composition for photodynamically inhibiting progression of non-alcoholic hepatitis, containing trisodium Chlorin e6 as an active ingredient.

### Advantageous Effects

According to one or more embodiments of the present invention, effects of treatment of obesity and non-alcoholic steatohepatitis can be exhibited.

### BRIEF DESCRIPTION OF THE DRAWINGS

FIG. 1 shows an experimental plan for anti-obesity analysis;

FIG. 2 shows the effect of PDT on the body weight of mice fed with high-fat diet;

FIG. 3 shows the effect of PDT on non-alcoholic steatohepatitis;

FIG. 4 shows the result of a toxicity test after PDT (systemic experiment);

FIG. 5 shows the result of a toxicity test after PDT (in-vivo experiment); and

FIG. 6 shows the result of a toxicity test after PDT (liver and white adipose tissue).

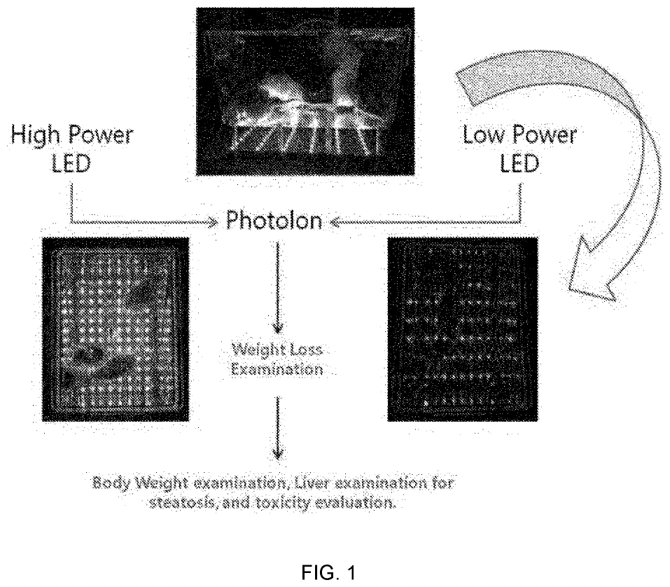

### DETAILED DESCRIPTION

A better understanding of constructions and effects of the present invention will be given with reference to the following embodiments. However, these embodiments are merely set forth to illustrate the present invention and are not to be construed as limiting the scope of the present invention.

The terms used herein are for describing the embodiments and are not intended to limit the present invention. In the present specification, the singular form also includes the plural form unless otherwise specified in the phrase. As used herein, ‘comprises’ and/or ‘comprising’ does not exclude the presence or addition of one or more other components.

Terms

As used herein, the term ‘treatment’ is meant to include eliminating a progressed disease or the cause of the disease, alleviating the disease or inhibiting further progression of the disease.

According to an embodiment of the present invention, a pharmaceutical composition for inhibiting obesity induced by a high-fat diet and progression of non-alcoholic steatohepatitis using a trisodium Chlorin e6 photosensitizer is disclosed.

According to the present invention, Chlorin e6 is an effective photosensitizer that has been used for a long time in various skin and cancer disease models. Chlorin is a large heterocyclic aromatic compound configured such that three pyrrole rings and one reduced pyrrole ring are connected by four methine bonds. Magnesium-containing chlorin is known as chlorophyll, and is the main photosensitive pigment in the chloroplasts of most plants, sea horses and cyanobacteria. Chlorin was introduced as a potential photosensitizer in PDT in the 1980s [References 11 and 12]. It has been found that a mixture of Chlorin e6 [Reference 13] and a hematoporphyrin derivative [Reference 14] is present in the cytoplasm and the cell membrane, such as a mitochondrial membrane. When a photosensitizer is specific to the surface antigen/receptor surface of target cells and is bound to antibodies or ligands, photosensitivity may increase. There have been results of Chlorin e6 acting on cancer cells along with monoclonal antibodies [References 15-17]. This is capable of increasing the selectivity of porphyrin photosensitivity by reacting specifically to the surface of cancer cells. The target cell for this interaction is the cell membrane [References 15 and 16]. It has been shown based on sensitivity to light exposure that intracellular organs such as nuclei and lysosomes [Reference 18] are likely to be much more sensitive targets [Reference 19]. As will be described later, according to the present invention, the use of Chlorin e6 as a photosensitizer is effective in treating obesity and non-alcoholic hepatitis.

The pharmaceutical composition for photodynamically inhibiting, alleviating or eliminating obesity according to an embodiment of the present invention (hereinafter referred to as a ‘pharmaceutical composition for the treatment of obesity’) contains trisodium Chlorin e6 as an active ingredient.

The pharmaceutical composition for photodynamically inhibiting, alleviating or eliminating non-alcoholic steatohepatitis according to another embodiment of the present invention (hereinafter referred to as a ‘pharmaceutical composition for the treatment of non-alcoholic steatohepatitis’) contains trisodium Chlorin e6 as an active ingredient.

In an embodiment of the present invention, trisodium Chlorin e6 contained in the pharmaceutical composition for the treatment of obesity and non-alcoholic steatohepatitis may have the following structure.

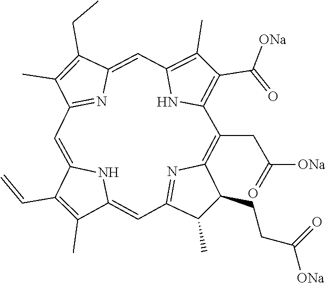

As another example, trisodium Chlorin e6 may have the following structure.

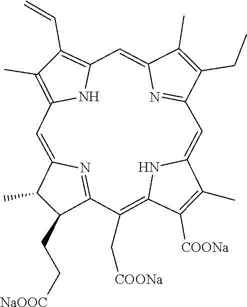

The above two trisodium Chlorin e6 structures are merely exemplary, and other forms of trisodium Chlorin e6 may also be used in the present invention.

Method of preparing pharmaceutical composition for the treatment of obesity according to embodiment of the present invention

The pharmaceutical composition for the treatment of obesity according to an embodiment of the present invention is prepared by mixing:

Chlorin e6 having the following structure

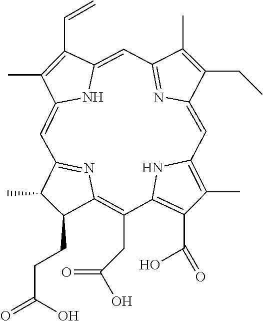 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C]

and represented by the molecular formula C₃₄H₃₆N₄O₆;

PVP (polyvinylpyrrolidone);

NaOH; and

HCl.

When the mixing process is performed in this way, trisodium Chlorin e6 is produced, resulting in a composite of trisodium Chlorin e6 and PVP (polyvinylpyrrolidone).

A pharmaceutical composition for the treatment of obesity according to another embodiment of the present invention may be a composite of trisodium Chlorin e6 and PVP (polyvinylpyrrolidone). This composite may be provided in the form of, for example, a powder. The pharmaceutical composition in a powder form for the treatment of obesity according to an embodiment of the present invention may be mixed with a saline solution and may thus be used for injection.

Method of preparing pharmaceutical composition for the treatment of non-alcoholic steatohepatitis according to embodiment of the present invention

The pharmaceutical composition for the treatment of non-alcoholic steatohepatitis according to an embodiment of the present invention is prepared by mixing:

Chlorin e6 having the following structure

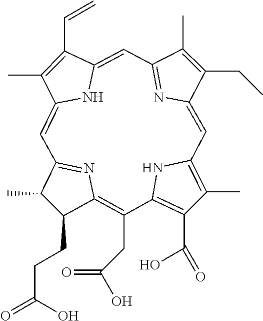 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C]

and represented by the molecular formula C₃₄H₃₆N₄O₆;

PVP (polyvinylpyrrolidone);

NaOH; and

HCl.

When the mixing process is performed in this way, trisodium Chlorin e6 is produced, resulting in a composite of trisodium Chlorin e6 and PVP (polyvinylpyrrolidone).

A pharmaceutical composition for the treatment of non-alcoholic steatohepatitis according to another embodiment of the present invention may be a composite of trisodium Chlorin e6 and PVP (polyvinylpyrrolidone). This composite may be provided in the form of, for example, a powder. The pharmaceutical composition in a powder form for the treatment of non-alcoholic steatohepatitis according to an embodiment of the present invention may be mixed with a saline solution and may thus be used for injection.

The experimental results for the embodiments of the present invention are described below.

Animal and Obesity Model Development

6-week-old C57 Black mice were purchased and were fed with normal diet for a week of environmental adaptation.

After feeding with normal diet for a week, these mice were randomly divided into 7 groups and tested. The body weight thereof was measured periodically (three times per week), and an anti-obesity experiment was conducted when the body weight was about 20% more than the initial body weight. The anti-obesity experiment was as follows.

For group 1, only high-fat diet (HFD) was provided and the body weight was periodically measured (HFD).

For group 2, only high-fat diet (HFD) was provided and the body weight was periodically measured, and the mice were placed in an LED-illuminated cage when the body weight thereof was about 20% or more than the initial body weight, followed by irradiation with low-level LED light.

For group 3, only high-fat diet (HFD) was provided and the body weight was periodically measured, and the mice were placed in an LED-illuminated cage when the body weight thereof was about 20% or more than the initial body weight, followed by irradiation with high-level LED light.

For group 4, only high-fat diet (HFD) was provided and the body weight was periodically measured, and when the body weight was about 20% or more than the initial body weight, trisodium Chlorin e6 (2.5 mg/kg) was dissolved in 200 μl of a saline solution and administered intraperitoneally. After intraperitoneal administration thereof, the mice were allowed to adapt to a dark room for 3 hr and then placed in an LED-illuminated cage, followed by irradiation with low-level LED light.

For group 5, only high-fat diet (HFD) was provided and the body weight was periodically measured, and when the body weight was about 20% or more than the initial body weight, trisodium Chlorin e6 (2.5 mg/kg) was dissolved in 200 μl of a saline solution and administered intraperitoneally. After intraperitoneal administration thereof, the mice were allowed to adapt to a dark room for 3 hr and then placed in an LED-illuminated cage, followed by irradiation with high-level LED light.

For group 6, only high-fat diet (HFD) was provided and the body weight was periodically measured, and when the body weight was about 20% or more than the initial body weight, trisodium Chlorin e6 (2.5 mg/kg) was dissolved in 200 μl of a saline solution and administered intraperitoneally.

The above group organization and treatment are summarized below for convenience of understanding.

Group Organization and Treatment

Group 1: high-fat diet (HFD)

Group 2: high-fat diet (HFD)+low-level LED irradiation

Group 3: high-fat diet (HFD)+high-level LED irradiation

Group 4: high-fat diet (HFD)+trisodium Chlorin e6 (2.5 mg/kg)+low-level LED irradiation

Group 5: high-fat diet (HFD)+trisodium Chlorin e6 (2.5 mg/kg)+high-level LED irradiation

Group 6: high-fat diet (HFD)+Chlorin e6 (2.5 mg/kg)

In the above experiments, the conditions of irradiation with LED light are as follows.

Low-level LED irradiation: 2.56 mW/cm², 5 min, 0.77 J/cm²

High-level LED irradiation: 4.96 mW/cm², 10 min, 2.98 J/cm²

Lipid Production and Examination of Non-alcoholic Steatohepatitis

After the treatment period was over, the mice were killed to measure body fat through a morphological examination. Moreover, after separating the liver from other organs, the shape of the liver was also observed. Fat accumulation in the liver was examined morphologically.

Toxicity Detection

The mice treated with Chlorin e6 were tested for potential toxicity by observing the shape of the internal organs through dissection after killing.

Results

1) Treatment of Obesity

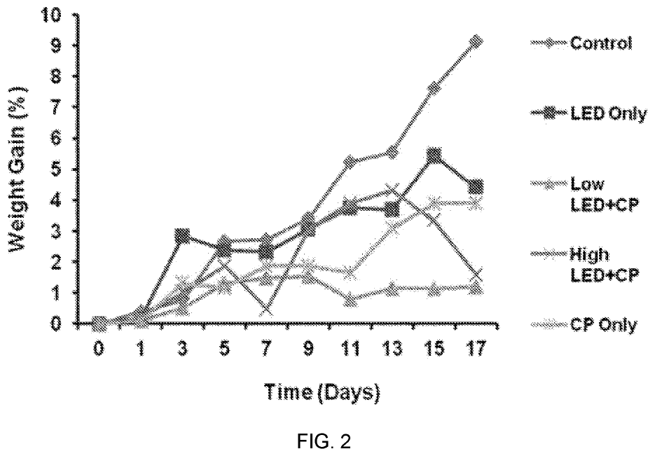

The treatment with normal diet and Chlorin e6 (Ce6) was determined to have the potential to reduce the body weight of the high-fat diet mice. The obvious symptom of obesity is uncontrollable weight gain, and the optimal treatment for obesity should be able to suppress weight gain and lower the body weight to the normal level. However, almost all drugs introduced to target weight control have failed, and desired results have been obtained by recommending strenuous exercise to patients. Therefore, in the present experiment, whether two groups treated with two concentrations of Chlorin e6, serving as a photosensitizer, together with the high- and low-power red LEDs, are able to reduce the percentage of weight gain compared to the group treated with HFD alone was evaluated. Consequently, it was confirmed that group 5 (high-level LED irradiation+2.5 mg/kg of Chlorin e6) was the most effective group and also that the weight loss was higher than group 4 (low-level LED irradiation+2.5 mg/kg of Chlorin e6) and group 1 (HFD), as shown in FIG. 2.

2) Treatment of Non-Alcoholic Steatohepatitis

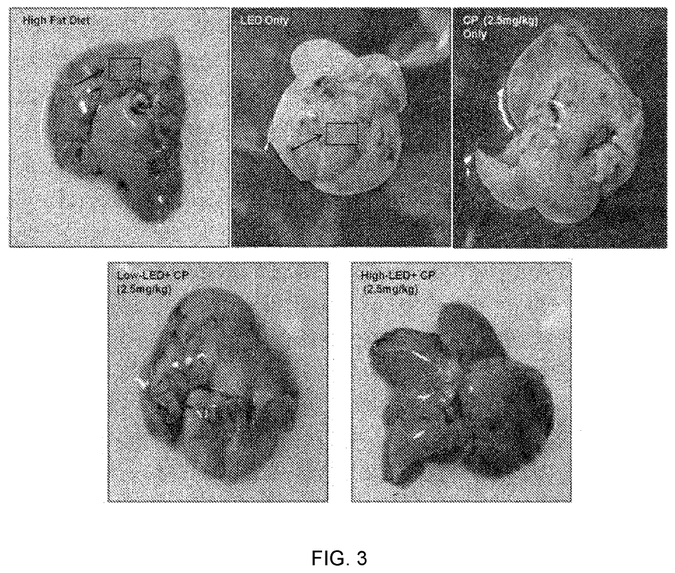

Fat accumulation in the liver, an important complication of obesity, is known as fatty liver disease or non-alcoholic steatohepatitis (NASH). Typically, non-alcoholic fatty liver disease (NAFLD) and non-alcoholic steatohepatitis have few symptoms. Abnormal health conditions, such as obesity, metabolic syndrome and type 2 diabetes, are likely to progress to NAFLD and NASH. Weight loss is recommended for the treatment of non-alcoholic fatty liver disease (NAFLD) and non-alcoholic steatohepatitis (NASH). Weight loss reduces liver fat, inflammation and fibrosis. There are no drugs approved for the treatment of NAFLD and NASH. However, in the present experiment, NASH was alleviated in group 5 (high-level LED irradiation+2.5 mg/kg of Chlorin e6) and group 4(low-level LED irradiation+2.5 mg/kg of Chlorin e6), as shown in FIG. 3, whereas in group 1 (HFD), the body weight was significantly increased. Meanwhile, the activity of PDT (Chlorin e6+light) is estimated to be able to prevent white adipose tissue from accumulating in the liver due to the change in gene expression.

3) Toxicity

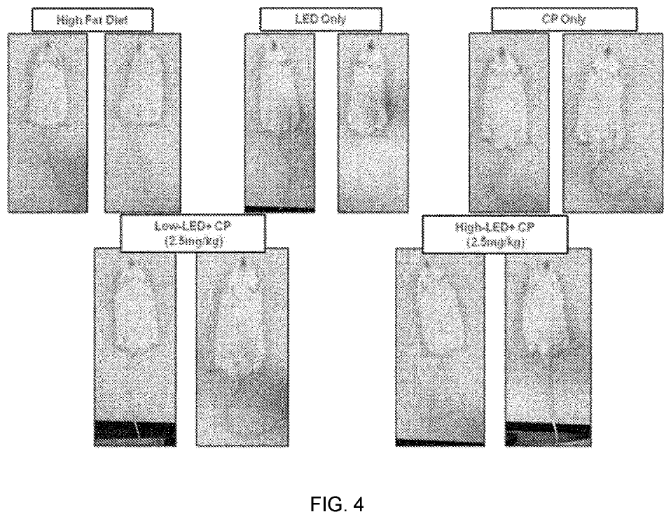

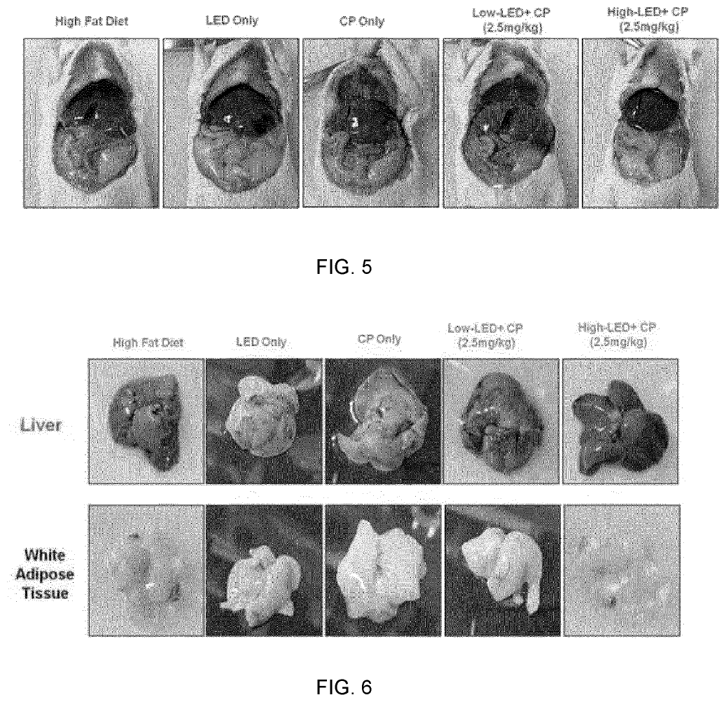

Toxicity was not observed during treatment with Chlorin e6 (Ce6) and LED irradiation in mice. The main concern in the development of medicaments or therapies is safety, and the safe use of medicaments means to effectively treat the disease with minimal or no toxicity in the body or parts of the body. Therefore, in the present experiment, the toxic effects of drugs and LED irradiation were examined. After the mice were dissected, the whole body and internal organs were examined in order to verify toxicity. In FIG. 4, there was no change in color throughout the mouse body, and the internal organs were intact; that is, no bleeding, color change or gangrene in the intestines was observed (FIG. 5). It was observed that the liver was normal other than the fatty liver observed in the groups treated with HFD alone and LED alone (FIG. 6).

As described above, although the present invention has been described with limited embodiments and drawings, the present invention is not limited to the above embodiments, and those skilled in the art to which the present invention pertains can make various modifications and variations from these descriptions. Therefore, the present invention should not be limited to the described embodiments, and should be defined not only by the claims below, but also by the equivalents thereof.

### REFERENCES

1. Statistics Canada. Canadian Community Health Survey 2004

2. Young L. R., Nestle M. The contribution of expanding portion sizes to the US obesity epidemic. Am. J. Public Health. 2002; 92:246-9.

3. Birmingham C. L., Muller J. L., Palepu A., Spinelli J. J., Anis A. H. The cost of obesity in Canada. CMAJ. 1999; 160:483-8.

4. Clark J. M., Brancati F. L., Diehl A. M. Non-alcoholic fatty liver disease. Gastroenterology. 2002; 122:1649-57.

5. Hubscher S. G. Role of liver biopsy in the assessment of non-alcoholic fatty liver disease. Eur. J. Gastroenterol. Hepatol. 2004; 16:1107-15.

6. Adams L. A., Lymp J. F., St. Sauver J., et al. The natural history of non-alcoholic fatty liver disease: A population-based cohort study. Gastroenterology.

7. Adams L. A., Angulo P. Treatment of non-alcoholic fatty liver disease. Postgrad. Med. J. 2006; 82:315-22.

8. Clinical guidelines on the identification, evaluation, and treatment of overweight and obesity in adults—the evidence report. National Institutes of Health. Obes. Res. 1998; 6(Supp12):515-2095. (Erratum in 1998; 6:461-2).

9. Sayiner M., Koenig A., Henry L., Younossi Z. M. Epidemiology of Non-alcoholic Fatty Liver Disease and Non-alcoholic Fatty liver disease in the United States and the Rest of the World. Clin. Liver. Dis. 2016; 20:205-214. [PubMed]

10. Kanwar P., Kowdley K. V. The Metabolic Syndrome and Its Influence on Non-alcoholic Fatty Liver Disease. Clin. Liver. Dis. 2016; 20:225-243. [PubMed]

11. Calzadilla Bertot L., Adams L. A. The Natural Course of Non-Alcoholic Fatty Liver Disease. Int. J. Mol. Sci. 2016; 17:pii: E774. [PMC free article] [PubMed]

12. Powell E. E., Cooksley W. G., Hanson R., Searle J., Halliday J. W., Powell L. W. The natural history of non-alcoholic fatty liver disease: a follow-up study of forty-two patients for up to 21 years. Hepatology. 1990; 11:74-80. [PubMed]

13. Caldwell S. H., Oelsner D. H., lezzoni J. C., Hespenheide E. E., Battle E. H., Driscoll C. J. Cryptogenic cirrhosis: clinical characterization and risk factors for underlying disease. Hepatology. 1999; 29:664-669. [PubMed]

14. Poonawala A., Nair S. P., Thuluvath P. J. Prevalence of obesity and diabetes in patients with cryptogenic cirrhosis: a case-control study. Hepatology. 2000; 32:689-692. [PubMed]

15. Teli M. R., James O. F., Burt A. D., Bennett M. K., Day C. P. The natural history of non-alcoholic fatty liver: a follow-up study. Hepatology. 1995; 22:1714-1719. [PubMed]

16. Younossi Z. M., Stepanova M., Negro F., Hallaji S., Younossi Y., Lam B., Srishord M. Non-alcoholic fatty liver disease in lean individuals in the United States. Medicine (Baltimore) 2012; 91:319-327. [PubMed].

17. Spikes J. D. Chlorins as photosensitizers in biology and medicine. J. Photochem. Photobiol. B 1990; 6(3):259-74.

18. Juzeniene A. Chlorin e6-based photosensitizers for photodynamic therapy and photodiagnosis. Photodiagnosis Photodyn. Ther. 2009 Jun; 6(2):94-6.

19. Henderson, B. W., and Dougherty, T. J. How does photodynamic therapy work—Photochem. Photobiol., 55: 145-157, 1992.

20. Bachor, R., Shea, C. R., and Hasan, T. Photosensitized destruction of human bladder carcinoma cells treated with chlorin e6-conjugated microspheres. Proc. Natl. Sci. USA, 88: 1580-1584, 1991.

21. Seroff, A. R., Ohuoha, D., Hasan, T., Bommer, J. C., and Yarmush, M. L. Antibody targeted photolysis: selective photodestruction of human T-cell leukemia cells using monoclonal antibody—chlorin e6 conjugates. Proc. Natl. Sci. USA, 83: 8744-8748, 1986.

22. Oseroff, A. R., Ara, G., Ohuoha, D., Aprille, i., Bommer, J. C., Yarmush, M. L, Foley, J., and Cincotta, L. Strategies for selective cancer photochemotherapy: antibody-targeted and selective carcinoma cells photolysis. Photochem. Photobiol., 46: 83-96,1987.

23. Goff, B. A., Bamberg, M., and Hasan, T. Photoimmunotherapy of human ovarian carcinoma cells ex vivo. Cancer Res., 51:4762-4767, 1991.

24. Robert, W. G., and Berns, M. W. In vitro photosensitization. Cellular uptake and subcellular localization of mono-L-aspartyl chlorin e6, chloro-aluminium-sulfonated phthalocyanine, and Photofrin II. Laser Surg. Med., 9: 90-101, 1989.

25. Alper, T. Cellular Radiobiology. Cambridge Univ. Press, Cambridge. 1979.

What is claimed is:

1. A method for obesity inhibition, comprising:
administering to a subject in need thereof a therapeutically effective amount of a composition comprising trisodium Chlorin e6.

2. The method of claim 1, wherein the composition is in a powder form.

3. The method of claim 1, wherein the trisodium Chlorin e6 has the following structure.

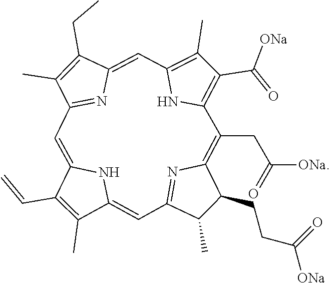

4. The method of claim 1, wherein the trisodium Chlorin e6 has the following structure.

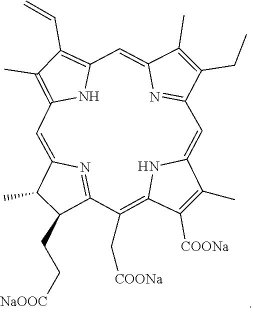

5. The method of claim 1, wherein the composition further comprises at least one selected from the group consisting of PVP (polyvinylpyrrolidone), NaOH; and HCl.

6. A method for photodynamically inhibiting progression of non-alcoholic hepatitis, comprising:
administering to a subject in need thereof a therapeutically effective amount of a composition comprising trisodium Chlorin e6.

7. The method of claim 6, wherein the composition is in a powder form.

8. The method of claim 6, wherein the trisodium Chlorin e6 has the following structure.

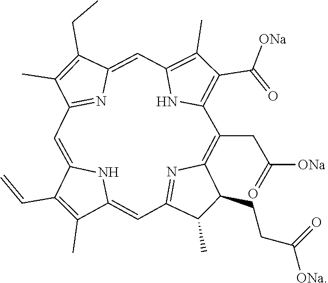

9. The method of claim 6, wherein the trisodium Chlorin e6 has the following structure.

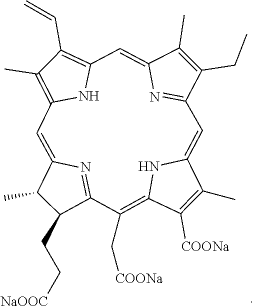

10. The method of claim 6, wherein the composition further comprises at least one selected from the group consisting of PVP (polyvinylpyrrolidone), NaOH; and HCl.
